(19) United States
(12) Patent Application Publication
(10) Pub. No.: US 2006/0078567 A1
(43) Pub. Date: Apr. 13, 2006

(54) Method of preventing acute pulmonary cell injury

(75) Inventors: Clifford S. Deutschman, Penn Valley, PA (US); Yoram Weiss, Tel-Aviv (IL)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: The Trustees of the University of Pennsylvania

(21) Appl. No.: 11/283,151

(22) Filed: Nov. 18, 2005

Related U.S. Application Data
(63) Continuation of application No. 10/150,054, filed on May 16, 2002.
(60) Provisional application No. 60/291,666, filed on May 17, 2001.

(51) Int. Cl. A61K 39/00

(52) U.S. Cl. 424/192.1

(57) ABSTRACT

The present invention discloses a method of preventing acute pulmonary cell injury associated with ALI or ARDS and a method of inhibiting an inflammatory response in pulmonary cells by increasing levels of HSP70 protein.

This application claims the benefit of priority from U.S. Provisional Application Ser. No. 60/291,666, filed May 17, 2001.

### BACKGROUND OF THE INVENTION

Sepsis and the related systemic inflammatory response syndrome (SIRS) and multiple organ dysfunction syndrome (MODS) are leading causes of death in critically ill surgical patients. The lung is the organ most often affected by these disorders, with damage taking the form of acute lung injury (ALI) and acute respiratory distress syndrome (ARDS) (Regel, et al. (1996) World J. Surg. 20:422-429). Recent studies indicate that mortality from ARDS is 30-98% (Baue, et al. (1998) Shock 10:79-89; Milberg, et al. (1995) JAMA 273:306-309). Most therapeutic modalities, including extracorporeal oxygenation, corticosteroids, prosta-glandins, surfactant, and nitric oxide, have failed to improve outcome (Fulkerson, et al. (1996) Arch. Intern. Med. 156:29-38). Supportive care, therefore, has been the mainstay of treatment for these patients.

The pathophysiology of ALI/ARDS remains unclear. However, a common finding is the recruitment and activation of inflammatory cells into the lung (Brennan, et al. (1995) Br. Med. Bull. 51:368-384; Rahman and MacNee (1995) Thorax 51:348-350). Recent data indicate that airway epithelial cells can also act as immune effectors by secreting proinflammatory mediators (Thompson, et al. (1995) Eur. Respir. 8:127-149). Once triggered, the inflammatory response damages the alveolar epithelium, leading to shedding of cells.

The lining layer of the adult lung contains two distinct types of cells. Type I cells are highly differentiated, flat, relatively quiescent and facilitate gas exchange. Type II cells are metabolically active, compact, have lamellar bodies containing surfactant and serve as progenitors for Type I cells (Simon and Paine (1995) J. Lab. Clin. Med. 126:108-118). After inflammatory injury, Type II cells undergo mitosis, differentiate into Type I cells and spread. Thus, preservation of Type II cells may facilitate recovery from ALI and ARDS.

Patients with ARDS are particularly vulnerable to secondary lung infections, including those induced by intracellular pathogens and viruses. Investigators have shown that uptake of adenoviruses in the lung is mediated by two receptors, the αᵥβ_{3/5} intregrins and the Coxsackie's-adenovirus receptor (CAR) (Bergelson (1999) Biochem. Pharmacol. 57: 975-979; Wickham, et al. (1993) Cell 73:309-319). The integrins modulate particle internalization, whereas CAR appears to be responsible for initial viral attachment. Integrin expression is increased in a number of cell types after many different injuries, including ARDS. This may predispose an individual to infection by enhancing viral uptake.

A number of natural mechanisms allow cells in virtually all organisms, from bacteria to mammals, to tolerate stresses that might otherwise be lethal. One such defense mechanism is the heat shock response. This involves the elaboration of a superfamily of proteins that are cytoprotective (Bellman, et al. (1995) J. Clin. Invest. 95:2840-2845; De Maio (1999) Shock 11:1-12). The 70-kD heat shock protein (HSP70) family is expressed in many organs and participates in protein preservation and repair. The expression of HSP70 has been detected in animal models of ischemia/reperfusion and endotoxemia. Under these conditions, HSP70 is induced in the lung, kidney, heart, and liver (Bellman, et al. (1995) J. Clin. Invest. 95:2840-2845; Klosterhalfen, et al. (1997) Shock 7:358-363; Marber, et al. (1995) J. Clin. Invest. 95:1446-1456; Tacchini, et al. (1993) Lab. Invest. 68:465-471; Wong and Wispe (1997) Am. J. Physiol. 17:L1-L9). Data indicate that heat treatment significantly improves outcome from phospholipid A1-mediated ALI, systemic-induced ARDS, and ischemia-reperfusion in lung isograft (Wong, et al. (1998) Am. J. Physiol. 275:L836-L841; Wong, et al. (1997) Am. J. Physiol. 272:L132-L138; Villar, et al. (1993) Am. Rev. Respir. Dis. 147:177-181; Hiratsuka, et al. (1998) J. Heart Lung Transplant 17:1238-1246). Also, heating an animal prior to administering an intravenous or inhaled toxic agent protects against lung injury and death (Rubero, et al. (1996) Am. J. Resp. Crit. Care Med. 153:A252; Villar, et al. (1994) Crit. Care Med. 22:914-922). Moreover, HSP70 gene transfection into rat lung isografts using an adenoviral vector provided a subsequent decrease in ischemia-reperfusion injury in the rat lung isografts (Hiratsuka, et al. (1999) Ann. Thorac. Surg. 67:1421-1427).

HSP70 expression is primarily transcriptionally controlled. In studies using a cecal ligation and puncture (CLP) model of sepsis in the rat, a significant down-regulation in the transcription of important hepatic genes was found (Andrejko, et al. (1998) Am. J. Physiol. 273:G1423-G1429; Deutschman, et al. (1997) Am. J. Physiol. 273:R1709-R1718). This down-regulation of transcription may lead to impaired protein synthesis contributing to sepsis-associated hepatic and pulmonary dysfunction (Weiss, et al. (2000) Shock 13:19-23; Andrejko and Deutschman (1997) Shock 7:164-169). Repletion of this protein synthetic capacity may have particular value during ALI because normal protein synthesis in Type II cells is likely essential for recovery.

A method of increasing the HSP70 levels in pulmonary cells to facilitate protection of pulmonary cells in ALI or ARDS is desired. The present invention is directed to this long felt need.

### SUMMARY OF THE INVENTION

The present invention provides a method of preventing pulmonary cell injury. The method comprises increasing the amount of pulmonary cell HSP70 protein levels to decrease ALI- or ARDS-associated signs or symptoms.

The invention further provides a method of inhibiting an inflammatory response in pulmonary cells. HSP70 protein levels are increased to cause a reduction in airway thickening, proteinaceous exudate, or neutrophilic infiltration.

### DESCRIPTION OF THE INVENTION

The present invention provides a method of preventing acute pulmonary cell injury. An increase in the levels of HSP70 protein facilitates protection of pulmonary cells in ALI or ARDS by inhibiting the inflammatory response.

Increases in HSP70 protein levels may be achieved by administering HSP70 protein directly to pulmonary cells or by expressing HSP70 protein from nucleic acid sequences encoding HSP70 which have been introduced into pulmonary cells.

In general, nucleic acid sequences encoding HSP70 or active portions thereof are introduced into eukaryotic expression vectors by conventional techniques. These vectors are designed to permit the transcription of the nucleic acid sequences encoding HSP70 in eukaryotic cells by providing regulatory sequences that initiate and enhance the transcription of the nucleic acid sequence and ensure its proper splicing and polyadenylation. Vectors containing the promoter and enhancer regions of the simian virus (SV)40 or long terminal repeat (LTR) of the Rous Sarcoma virus and polyadenylation and splicing signal from SV(40) are readily available (Mulligan, et al. (1981) Proc. Natl. Acad. Sci. USA 78:1078-2076; Gorman, et al. (1982) Proc. Natl. Acad. Sci. USA 79:6777-6781).

Whole or an active portion of HSP70 protein or fusion proteins comprised of HSP70 may be used in the methods of the present invention. For example, nucleic acid sequences encoding a fusion protein, consisting of a molecule comprising a portion of the HSP70 sequence plus a non-HSP70 sequence, may be produced using well-known methods, for example WO 91/05047. Thus, further modifications of HSP70 include the generation of chimeric molecules containing portions of the HSP70 sequences attached to other molecules whose purpose is to affect solubility, pharmacology or clearance of the resultant chimeras. Such chimeras may be produced either at the gene level as fusion proteins or at the protein level as chemically produced derivatives. Molecules that may be used to form chimeras include, but are not limited to, proteins such as serum albumin, heparin, or immunoglobulin, polymers such as polyethylene glycol or polyoxyethylated polyols, or proteins modified to reduce antigenicity by, for example, derivatizing with polyethylene glycol. Suitable molecules are known in the art and are described, for example, in U.S. Pat. Nos. 4,745,180, 4,766,106 and 4,847,325 and references cited therein. Additional molecules that may be used to form derivatives of the biological compounds or fragments thereof include protein A or protein G (WO 87/05631; Bjorck, et al. (1987) Mol. Immunol. 24:1113-1122; Guss, et al. (1986) EMBO J. 5:1567-1575; Nygren, et al. (1988) J. Molecular Recognition 1:69-74).

The level of expression of the nucleic acid sequences encoding HSP70 or HSP70 fusion proteins may be manipulated by using promoters that have different activities, for example, the baculovirus pAC373 can express nucleic acid sequences at high levels in S. frungiperda cells (Summers and Smith, In: Genetically Altered Viruses and the Environment (B. Fields, et al, eds.) vol. 22 pp. 319-328, Cold Spring Harbour Laboratory Press, Cold Spring Harbor, N.Y., 1985) or by using vectors that contain promoters amenable to modulation, for example the glucocorticoid-responsive promoter from the mouse mammary tumor virus (Lee, et al. (1982) Nature 294:228).

The cloning vector comprises the nucleic acid sequences encoding HSP70 for expression in a suitable host. The nucleic acid is operably linked to a promoter sequence such that HSP70 protein may be expressed in effective amounts. The promoter sequence may be sequences that control the expression of genes of prokaryotic or eukaryotic cells and their viruses and combinations thereof. Examples of promoter sequences include, but are not limited to, the lac system, the trp system, the tac system, the trc system, major operator and promoter regions of phage lambda, the control region of fd coat protein, CMV immediate early, HSV thymidine kinase, the early and late promoters of SV40, promoters derived from polyoma, adenovirus, retrovirus, baculovirus and simian virus, the promoter for 3-phosphoglycerate kinase, the mouse metallothionein-I promoter, the promoters of yeast acid phosphatase, the promoter of the yeast alpha-mating factors and other promoters known to control expression of genes in prokaryotic or eukaryotic cells or their viruses.

The vector may also contain a ribosome binding site for translation initiation and a transcription terminator. The vector may also contain enhancer sequences to increase transcription of a nucleic acid encoding the HSP70 protein to effective amounts. Enhancers are cis-acting elements of DNA, usually from about 10 to 300 bp, that act on a promoter to increase its transcription. Examples include the SV40 enhancer on the late side of the replication origin (bp 100 to 270), a cytomegalovirus early promoter enhancer, a polyoma enhancer on the late side of the replication origin, and adenovirus enhancers.

In addition, some vectors contain selectable markers, such as the gpt or neo bacterial genes that permit isolation of cells, by chemical selection, that have stable, long-term expression of the vectors, and therefore the nucleic acid sequences, in the recipient cell. Alternatively, the vector may have no selectable marker as described in U.S. Pat. No. 6,265,218, herein referenced in its entirety. The vectors may be maintained in the cells as episomal, freely replicating entities by using regulatory elements of viruses such as papilloma or Epstein-Barr. Alternatively, one may also produce cell lines that have integrated the vector into genomic DNA. Both of these types of cell lines produce the gene product on a continuous basis. Cell lines may be produced that have amplified the number of copies of the vector, and therefore of the nucleic acid sequences as well, to create cell lines that can produce high levels of HSP70 or HSP70 fusion protein.

The HSP70 proteins may then be isolated and purified by standard methods including chromatography. For example, ion exchange, affinity, and sizing column chromatography, high performance liquid chromatography; centrifugation, differential solubility; or by any other standard technique for the purification of proteins. The purified HSP70 or HSP70 fusion protein may then be used for direct delivery to pulmonary cells in accordance with methods of the present invention.

The transfer of DNA into eukaryotic, in particular human or other mammalian cells is well-known in the art. The vectors are introduced or transfected into the recipient cells as pure DNA by, for example, precipitation with calcium phosphate or strontium phosphate, electroporation, lipofection, DEAE dextran, microinjection, protoplast fusion, or pellet guns. Alternatively, the cDNA can be introduced by infection with virus vectors such as retroviruses, adenoviruses or Herpes virus. Virus vectors containing nucleic acid sequences encoding HSP70 may be employed as aerosolized suspensions as disclosed in U.S. Pat. No. 6,344,194.

The expression of the nucleic acid sequences may be monitored in the recipient cells 24 to 72 hours after introduction. Current gene expression from viral or non-viral gene delivery systems are typically too inefficient or transient to offer clinical benefit for treatment of most lung diseases (Vadolas, et al. (2002) Pulm. Pharmacol. Ther. 15(1):61-72), however transient expression of HSP70 proteins is preferred for preventing acute pulmonary lung cell injury because ALI and ARDS are transient diseases.

An effective amount of HSP70 protein is defined as an amount which decreases the signs or symptoms of ADI or ARDS. Examples of ADI or ARDS signs and symptoms include, but are not limited to, pulmonary edema, airway thickening, atelectasis, honeycombing, hypoxemia, tachypnea, and neutrophilic infiltration.

The preferred route of administration of HSP70 protein or HSP70 encoding nucleic acid sequences is in the aerosol or inhaled form. The HSP70 protein or HSP70 encoding nucleic acid sequences, combined with a dispersing agent, or dispersant, can be administered in an aerosol formulation as a dry powder or in a solution or suspension with a diluent as is well-known in the art, for example U.S. Pat. Nos. 6,169,068 and 6,334,999.

In general, the HSP70 protein, or an active fragment or derivative thereof is introduced into the subject in the aerosol form in an amount between 0.01 mg per kg body weight of the mammal up to about 100 mg per kg body weight of said mammal. One of ordinary skill in the art may readily determine a volume or weight of aerosol corresponding to this dosage based on the concentration of HSP70 protein in an aerosol formulation.

Systems of aerosol delivery, such as the pressurized metered dose inhaler and the dry powder inhaler are disclosed in Newman, S. P., Aerosols and the Lung, Clarke, S. W. And Davia, D. editors, pp. 197-22 and can be used in connection with the present invention.

In accordance with methods of the present invention, an animal model of ARDS, cecal ligation and double puncture (2CLP) in rats, was used to demonstrate the use of viral-mediated overexpression of HSP70 for protection of pulmonary cells in ALI/ARDS. Hypoxemia, tachypnea, and neutrophilic infiltration characterize clinical ALI/ARDS. The lung develops an ARDS-like phenotype resulting from an intraperitoneal inflammatory process. Changes in arterial oxygen tension (PaO₂), respiratory rate, bronchoalveolar lavage protein content, and myeloperoxidase concentrations over time after 2CLP and sham-operated (SO) were evident. Compared with sham operation, 2CLP resulted in a 50% increase in respiratory rate and a 50% decrease in PaO₂. Myeloperoxidase concentrations after 2CLP were 25% above baseline, significantly different from SO, and indicative of neutrophilic infiltration. Bronchoalveolar lavage protein content after 2CLP doubled, reflecting capillary leak. Hematoxylin and eosin-stained sections confirmed the presence of a significant neutrophilic infiltrate and also revealed septal thickening, increased cellularity, and proteinaceous exudate, typical of the lung pathology observed during clinical ALI/ARDS. Within each lung there was a spectrum of disease, ranging from mild injury to severe atelectasis and honeycombing. Minor levels of atelectasis were noted in lungs from SO and nonoperated animals.

To determine the extent to which adenoviruses were taken up by pulmonary epithelial cells after 2CLP or sham operation, Green Fluorescent Protein (GFP) fluorescence was examined. Data were obtained from three nonoperated animals that were administered phosphate-buffered saline (PBS) without virus, three nonoperated animals that were administered GFP-expressing virus, three SO animals that were administered GFP-expressing virus, and five 2CLP animals that were administered GFP-expressing virus. Ten fields in each section were evaluated. Relative to nonoperated and SO controls, there was a statistically significant (P<0.001) increase in GFP expression in the lungs after 2CLP. The majority of this expression was observed in small airways and surrounding alveoli with little expression in the trachea, bronchi, and bronchioles. GFP fluorescence was most prominent in mildly to moderately diseased areas of 2CLP lung but was not observed in severely diseased regions. No GFP expression was noted in heart, liver, and kidney sections examined after 2CLP.

Administration of 3×10¹¹ or 3×10¹² plaque-forming units (pfu) resulted in comparable levels of GFP expression. However, 48 and 72 hours after 3×10¹² viral pfu was administered, a degree of viral-mediated lymphocytic infiltration was observed. Despite similar levels of GFP expression, little lymphocytic infiltration was observed after administration of the lower viral dose.

Light microscopy indicated that most GFP expression after 2CLP occurred in Type II cells. However, 48 hours after adenovirus administration and 2CLP, fluorescence was detected lining the alveoli. This is consistent with either secretion of GFP or with GFP expression within Type I cells. Some Type I cells appeared to be GFP-positive. To determine more precisely which cells were taking up adenoviruses and expressing gene product, electron microscopy and semithin section light microscopy were employed. One nonoperated animal was administered PBS without virus, one nonoperated animal received 10¹¹ pfu LacZ-expressing adenovirus, two SO operated animals were given a like dose of LacZ-expressing viruses, and four 2CLP animals were administered LacZ-expressing virus. Electron and semithin section light microscopy were performed 48 hours after intervention. The LacZ gene product 48 hours after 2CLP localized primarily to lamellar body-containing Type II cells, although staining was also observed in some Type I cells. Similar results were obtained in all four 2CLP animals and in the two SO rats. Viral uptake and LacZ expression in nonoperated animals was not sufficient to assess localization. Semithin sections examined under 60×magnification confirmed that Type II cells were the primary sites of expression although again, some Type I cells contained LacZ gene product. Some expression was also observed in interstitial macrophages.

Enhanced GFP expression after 2CLP may have been due to an up-regulation of one or all of the receptors involved in adenovirus attachment and internalization. To determine changes in the relative abundance and location of these receptors after 2CLP, immunohistochemistry was performed. These studies demonstrated an increase in αᵥβ_{3/5} integrin and CAR expression after 2CLP relative to SO or nonoperated controls. Treatment with only secondary antibodies followed by avidin, biotinylated alkaline phosphatase and NBT-BCIP did not result in any staining.

Increased apical abundance may result from either augmented production or from translocation of intracellular or basal membrane receptors. Therefore, protein abundance in lung homogenate was determined using immunoblotting. Data from autoradiograms were quantified using laser densitometry. Relative to both nonoperated controls and SO animals, a statistically significant increase in both αᵥβ₃ integrin and CAR abundance was observed after 2CLP. αᵥβ₃ integrin concentrations increased at 3, 6, 16, 24, and 48 hours, whereas CAR abundance was significantly greater only at 2 and 6 hours. No change was noted in αᵥβ₅ integrin concentrations.

Viral-mediated overexpression of HSP70 was then examined. Adenoviruses expressing HSP70 were administered via the trachea to rats after 2CLP, sham operated and unoperated controls. As with GFP, HSP70 expression was prevalent in Type II cells. Histologic changes in lungs of rats subjected to 2CLP then treated with virus expressing HSP70 revealed marked differences when compared to rats administered a virus expressing GFP. HSP70 overexpression decreased the number of inflammatory cells trapped in the alveolar interstitium, reduced the proteinaceous exudate in the alveolar air spaces and limited airway thickening. Accordingly, the lung appeared comparable to an untreated lung. Moreover, outcome was significantly affected. Survival of rats treated with virus expressing HSP70 was 62% ( 52/84), while rats treated with virus expressing GFP was 31% ( 29/94) The invention is further illustrated by the following, non-limiting examples.

### EXAMPLE 1

Induction of Sepsis-Acute Lung Injury

During methoxyflurane anesthesia, fulminant sepsis was induced in male Sprague-Dawley rats (Charles River, Boston, Mass.; weight, 250-275 grams) using cecal ligation and double puncture (2CLP) with an 18-gauge needle (Andrejko and Deutschman (1997) Shock 7:164-169). Sham-operated (SO) animals and animals not undergoing abdominal surgery served as controls. After the procedure, rats underwent fluid resuscitation with 40 ml/kg of subcutaneously injected sterile saline. Animals were awakened and allowed free access to water and food. Fluid resuscitation was repeated every 24 hours until they were sacrificed. At 24, 48, and 72 hours after surgery, respiratory rate was determined, and animals were reanesthetized with 40 mg/kg intraperitoneal pentobarbital. Blood for arterial oxygen tension (PaO₂) analysis was obtained, and the rats were sacrificed via exsanguination. A separate group of animals were sacrificed at 0 (three animals), 3, 6, 16, 24, and 48 hours after 2CLP (27 animals) or SO (10 animals). In these animals, lung tissue was prepared for immunoblot analysis of CAR and integrin expression.

In one group of 62 animals (3 nonoperated controls, 47 subjected to 2CLP, 12 SO animals), bronchoalveolar lavage was performed just after they were sacrificed. Lungs were infused with 1.5 ml of room-temperature phosphate-buffered saline (PBS) until fully distended. Fluid was withdrawn and saved. This process was repeated three times, volumes were pooled and recorded, and protein content was determined using the Bradford kit according to manufacturer's instructions (Pierce, Rockford, Ill.).

Myeloperoxidase activity, an index of neutrophil content in tissue, was determined using modifications of methods well-known in the art, for example Calderon, et al. ((1990) J. Biochem. Biophys. Meth. 20:171-180). Briefly, lung tissue obtained from a second group of 62 animals, immediately after they were sacrificed, was excised, homogenized in 0.1 M potassium phosphate buffer, centrifuged, resuspended, sonicated, and recentrifuged. The supernatant was treated with o-dianisodine and H₂O₂, and changes in absorbance over a 3-minute time period at 460 nm were observed.

Just after sacrificing, lungs were removed en bloc from a third group of 62 rats, inflated and fixed overnight in 10% neutral-buffered formalin, sliced sagittally, paraffin-embedded, cut into 5 μm sections, and stained with hematoxylin and eosin. Light microscopy was performed.

### EXAMPLE 2

Virus Administration

Recombinant E1-deleted adenoviruses expressing green fluorescent protein (GFP) or bacterial LacZ genes or HSP70 with a cytomegalovirus promoter were used in initial analyses. Vector was resuspended within 1 hour of administration to avoid a decline in viral titer.

Viral particles were suspended in PBS and injected via a 24-gauge tracheal cannula inserted immediately after sham operation (12 animals) or 2CLP (76 animals), or into a group of six nonoperated animals. Over 10 minutes, one of two viral doses, either 3×10¹² or 3×10¹¹ viral plaque-forming units (pfu) in PBS with a total volume of 300 μl, were delivered in three divided aliquots. Three control animals received PBS without virus. Animals were allowed to recover and were sacrificed at 0, 24, and 48 hours after injection.

### EXAMPLE 3

Detection of Viral Uptake into Lung Tissue

Green fluorescent protein-treated lung tissue excised from rats was formalin-fixed, paraffin-embedded, and sectioned as described above. GFP expression was determined on representative 5-μm sections using fluorescence microscopy. Three representative sections from each of three animals at each time point were chosen for examination. Total fluorescence per high-power field was determined electronically. In each representative section, fluorescence from 10 different high-power fields was quantified. The number of counts per high-power field was averaged for each animal. Fluorescence levels per high-power field for three SO and three 2CLP animals at each time point were averaged, and standard errors were determined. Heart, kidney, and liver tissue was excised and fixed, and GFP expression in these tissues was determined using similar methodology.

### EXAMPLE 4

Pathologic Examination

A pathologist unaware of treatment evaluated sections. Hematoxylin- and eosin-stained sections were examined for evidence of congestion, atelectasis, inflammatory cell accumulation, alveolar edema, hyaline membrane formation, and lymphocytic infiltration. Fluorescence microscopy was used to evaluate sections for the presence and distribution of staining. GFP was evaluated using a dual-pass filter that transmitted the appropriate wavelengths of light to excite both rhodamine and fluorescein isothiocyanate conjugate and eliminated autofluorescence. Sections were evaluated and scored on the basis of ARDS-like pathology and the number of GFP-positive cells per 40× high-powered field (three fields per animal).

### EXAMPLE 5

Immunohistochemical Detection of Coxsackie-Adenovirus Receptor, α_{V}β_{3/5} Integrins, and HSP70

Lung sections were deparaffinized, rehydrated, and heat-treated in citrate buffer (Antigen Unmasking Solution, Vector Labs, Burlingame, Calif.). In animals not treated with virus, sections were washed with PBS, incubated with a primary mouse antibody to human CAR or primary goat polyclonal antibodies to αᵥβ₃ or αᵥβ₅ integrins (Chemicon International, Temicula, Calif.) or rat HSP70 followed by secondary goat-antimouse IgG or rabbit-antigoat IgG (Vector Labs). Human CAR is known to cross-react with both rat and mouse antigens. After washing, sections were treated with avidin and biotinylated alkaline phosphatase, followed by nitroblueterazolium-5-bromo, 4-chloro, 3-indolyphosphate (NBT-BCIP)(Boerhinger, Mannheim, Germany).

### EXAMPLE 6

Detection of Coxsackie-Adenovirus Receptor and αᵥβ_{3/5} Integrin Proteins Using Immunoblotting

In a separate group of rats subjected to 2CLP (27 animals) or SO (10 animals), total lung protein was isolated at 0, 3, 6, 16, 24, and 48 hours, and immunoblot analysis was performed (Weiss, et al. (2000) Shock 13:19-23). Bradford assays were conducted according to manufacturer's instructions (Pierce, Rockford, Ill.) and was used to determine homogenate protein concentration. Human CAR and αᵥβ_{3/5} integrins were detected using the same primary and secondary antibodies described above. Detection was performed via enhanced chemiluminescence (Amersham Biotech, Buckinghamshire, United Kingdom). Blotting was performed on purified integrin (Chemicon International, Burlingame, Calif.) and CAR samples to assure that binding was specific. To further assure even loading, blots were also probed with an antibody to GAPHD (Chemicon International). Concentrations were quantified with scanning laser densitometry (Mulligan (1993) Science 260:926-932). Densities for CAR or the integrins were divided by the density for GAPHD at the same time point in the same blot. This corrected density was then normalized to the density at T₀ on the same blot.

### EXAMPLE 7

Electron Microscopic and Semithin Section Determination of LacZ Expression

Lungs from animals injected with LacZ containing adenovirus were removed en bloc. Tissue was processed using methods well-known in the art (Byrne, et al. (1994) Development 120:2369-2383). The lung tissue was minced into 0.5-cm cubes, incubated with fresh X-Gal at room temperature for 4 hours, washed with PBS, fixed with 2.5% glutaraldehyde for 4 hours, and washed with sodium cacodylate buffer. Samples were trimmed under a dissecting microscope, stained en bloc with 1% uranyl acetate for 1 hour in the dark, dehydrated with alcohol, embedded in epoxy LX-112, and polymerized at 70° C. for 72 hours. Fixed tissue was sectioned to a thickness of 8-nm for electron microscopy. Semithin sections (1.5 μm) were cut with a histo-diamond knife, stained briefly with toluidine blue, and examined by light microscopy.

### EXAMPLE 8

Statistical Analysis

Analysis of variance with the Bonferroni correction (P<0.05) was used to identify significant differences between SO and 2CLP and over time.

1-3. (canceled)

4. A method of protecting a lung of a patient from pulmonary cell injury, said method comprising the administration to said patient of an effective amount of a nucleic acid comprising a nucleic acid sequence encoding at least one HSP70 protein.

5. The method of claim 4, wherein said HSP70 protein is selected from the group consisting of an active fragment of HSP70 and an HSP70 fusion protein.

6. The method of claim 4, wherein said pulmonary cell injury is selected from the group consisting of an acute injury and a chronic injury.

7. The method of claim 4, wherein said pulmonary cell injury is associated with a disorder selected from the group consisting of acute lung injury (ALI), acute respiratory distress syndrome (ARDS), pulmonary inflammation, and pulmonary sepsis.

8. The method of claim 4, wherein said nucleic acid comprises a nucleic acid vector.

9. The method of claim 4, wherein said nucleic acid comprises a vector selected from the group consisting of a eukaryotic vector and a viral vector.

10. The method of claim 9, wherein said viral vector is an adenoviral vector.

11. The method of claim 9, wherein said adenoviral vector further comprises a viral particle.

12. A method of decreasing a sign or symptom in a patient of a disorder selected from the group consisting of ALI and ARDS, said method comprising the administration to said patient of an effective amount of a nucleic acid comprising a nucleic acid sequence encoding at least one HSP70 protein.

13. A method of inhibiting an inflammatory response in a pulmonary cell of a patient, said method comprising the administration to said patient of an effective amount of a nucleic acid comprising a nucleic acid sequence encoding at least one HSP70 protein.
